(19) United States
(12) Patent Application Publication
(10) Pub. No.: US 2020/0375808 A1
(43) Pub. Date: Dec. 3, 2020

(54) SWAB ASSEMBLY AND SYSTEM

(71) Applicant: Paulo Roberto Jannotti Newlands, Rio de Janeiro (BR)

(72) Inventor: Paulo Roberto Jannotti Newlands, Rio de Janeiro (BR)

(21) Appl. No.: 16/845,356

(22) Filed: Apr. 10, 2020

Related U.S. Application Data
(63) Continuation-in-part of application No. 16/430,305, filed on Jun. 3, 2019, now abandoned.

(51) Int. Cl. A61F 13/38; A61F 13/551; B65D 25/04; A45D 40/28

(52) U.S. Cl. CPC A61F 13/38; A61F 13/385; A45D 40/28; B65D 25/04; A61F 13/551

(57) ABSTRACT

The present invention provides for an environmentally friendly system and associated methods for a user to utilize a disposable stem and a removable, reusable handle member. Specifically, the present invention provides for a method of manufacturing and a system of using a stem having a proximal end and a distal end, with an absorbent material fixed to the distal end, while the proximal end is configured to be interconnected with the removable handle member for a user to utilize the disposable swab system for many purposes, such as cleaning a user's ears and for other general use.

### CLAIM OF PRIORITY

The present invention is a continuation in part of a previously filed, now pending application having U.S. application Ser. No. 16/430,305 and a filing date of Jun. 3, 2019, the contents of which is explicitly incorporated herein by reference.

### FIELD OF THE INVENTION

The present invention provides for a system and method for producing a unique disposable swab for general purpose use. Specifically, a disposable stem having an absorbent material affixed to one end is configured to be concentrically interconnected to a removable handle member for proper utilization by a user.

### DESCRIPTION OF THE RELATED ART

Swabs have been used as multi-purpose articles of personal hygiene, including but not limited to cosmetic, medical, and general care uses. Swabs have been produced with plastic stems, but paper stem swabs have progressively dominated the market. Presently, swabs may be constructed with an elongated stem composed of plastic or cellulose products, including cotton pads affixed directly to both ends of the stem.

Recently, restrictions are being imposed globally against non-environmentally friendly products, including plastic swabs. In response to this movement, the production of paper swabs has mitigated the detrimental effects of plastic-produced swabs. Nonetheless, an improved solution is needed in this field to overcome the extensive production of paper, leading to deforestation and landfill accumulations.

A system and associated methods of production are needed to produce conventional swabs with the purpose of reducing the ecological footprint of waste materials disposed in nature. Accordingly, an aim of the present invention is to provide a new, disposable swab system that will reduce the ecological footprint of current cotton swabs. In this present invention, a swab, or stem may have one end with an absorbent material, such as cotton, and a second end capable of being interconnected to a removable, and re-usable handle member. The present invention can reduce the amount of manufacturing materials used in producing swabs while also providing a re-usable and environmentally friendly removable handle member.

### SUMMARY OF THE INVENTION

The present invention meets the existing needs of mitigating the detrimental effects to the environment caused by cotton swabs currently on the market. Accordingly, in initially broad terms, an apparatus or system directed to the present invention comprises a disposable stem, having a single end covered by a cleaning absorbent material and a second end capable of forming an interconnected relationship to a removable and reusable handle member for comfortable and convenient utilization by a user. Additionally, a primary purpose of the removable handle is to decrease the length of the stem, providing a means of reducing the materials involved in the manufacture of the present invention and contributing to an ecologically and environmentally friendly product. A user may connect the removable handle member to the stem having the affixed absorbent material and use the present invention for cleaning, medical, cosmetic, and general care. The user can then dispose of the single-end swab, and re-use the removable handle for further use with other stems with the attached absorbent material.

Accordingly, in at least one embodiment of the present invention, a disposable swab system may comprise a stem having a proximal end, a distal end, and an absorbent material disposed on an exterior of the distal end. The disposable swab system will further comprise a removable handle member dimensioned and configured to be in an interconnected relationship with one end of the stem, the proximal end. The stem may be hollow, solid, or a combination thereof. Additionally, the stem and absorbent material may be composed of cellulose materials, such as paper, cotton, linen, and other plant fibers that are environmentally friendly. The removable handle member may be introduced into a proximal end of the stem, resulting in an interconnected relationship. The interconnected relationship comprises a mating, preferably concentric relation, between the removable handle member and the stem. Once interconnected, the disposable swab system is in an operative position to be utilized by a user.

For example, in one preferred embodiment the stem is at least partially hollow. As such, the removable handle member can be partially or fully inserted into the open, proximal end of the stem, providing a means of accomplishing the mating between the stem and the removable handle member. In this preferred embodiment, the removable handle member may comprise a diameter less than the diameter of the stem. Also, the removable handle member may further comprise at least one retention or grip ring to further stabilize and/or retain the interconnected relationship. Additionally, because the stem is at least partially hollow, the removable handle member may be at least partially solid in this preferred embodiment to ensure the disposable swab system has sufficient rigidity and density to be property utilized by a user when in an operative position. The removable handle member may comprise a circular end, a cylindrical body and a conic end. In this preferred embodiment, the conic end of the removable handle member may be inserted into the proximal end of the stem. A predetermined portion of the cylindrical body will be disposed both inside and outside the stem.

Furthermore, the stem may comprise an obstructive element located in the interior of the distal end having the affixed absorbent material. The obstructive element, including but not limited to a plug or disc-shaped structure, will act as a protective structure. Specifically, the plug will function as a physical limitation for the reach of the removable handle member within the stem, ensuring the removable handle member does not surpass the limits of the absorbent material affixed at the exterior of the distal end. This provides for user safety when utilizing the disposable swab system in an operative position. The plug may be made of cellulose and environmentally friendly materials.

Another obstructive element, or shoulder structure, may be configured on the removable handle member. Accordingly, the removable handle member may comprise an enlarged, thicker section opposite the end inserted into the stem. When the removable handle member is inserted within the stem, the enlarged, thicker section will be a shoulder structure limiting the reach of the removable handle member within the stem. Additionally, because the enlarged, thicker section will not be inserted into the stem, it also functions as a cover for the portion of the removable handle member disposed outside the stem.

In an alternative embodiment of the disposable swab system, the stem and the removable handle member may have an inverse relationship. In this embodiment, the stem is at least partially solid. As such, the proximal end of the stem will be inserted within the removable handle member, providing the necessary means of accomplishing the concentric mating between the stem and the removable handle member. In this inverse relationship, the stem may comprise a diameter less than the diameter of the removable handle member. Additionally, the removable handle member can comprise at least one retention ring on its interior, disposed and structured for retaining engagement with the stem. The at least partially solid stem will now provide the necessary rigidity and density to be conveniently utilized by a user when the disposable swab system is in an operative position. As in the previous embodiment, the interior of the distal end of the stem may comprise an obstructive element or plug to act as a limitation for the disposable swab system.

The present invention may also be incorporated into a packaging system for storing and using the disposable swab system by a user. In one preferred embodiment, the packaging system comprises a container. The container may comprise an open top, a closed bottom, and a plurality of individual compartments. The container may also comprise a cover movably disposed in covering relation the open top. The packaging system further comprises a plurality of stems, each stem comprising a proximal end and a distal end, with an absorbent material fixed at each distal end of the plurality of stems. Each stem will be housed in an individual compartment within the container. The individual compartments will have a diameter less than a diameter of the stems for proper storage.

The packaging system will also comprise the removable handle member, which may be housed in a separate compartment integrated within the container. Importantly, the plurality of stems will be positioned in an upright position. The upright position comprises the proximal end being disposed adjacent to the open top in an exposed and accessible position relative to interconnection with the removable handle member. The distal end with the absorbent material will be adjacent to the closed bottom. This upright position will allow a user to grab the removable handle member and easily introduce the handle into the hollow end of the proximal stem, without even having to remove the stem from the container. This provides for easy and comfortable utilization of both the packaging and use system, and the utilization of the disposable sway system. In line with the environmental objectives of the present invention, the container may be composed of cellulose material, carton material, recyclable material, or any other material.

Additionally, the present invention also incorporates a method for manufacturing the disposable swab system. Accordingly, an important step in this method of manufacturing is to form an obstructive element within the distal end of the stem to act as a stopper limitation for the removable handle member's reach within the stem. The obstructive element may be composed of a variety of material. In one embodiment, environmentally friendly material such as paper or cellulose material is used. Accordingly, a desired portion of solid cylindrical paper will be introduced into the hollow, distal end of the stem, which will then be affixed in the obstructive position, by means of glue or any other mechanism to permanently fix the obstructive element. Then, the desired portion can be separated from the rest of the longer, cylindrical material, resulting in the obstructive element disposed within the distal end.

In one embodiment, the obstructive element can take the shape of a disc or plug having a diameter less than an internal diameter of the stem. Now that the obstructive element is firmly positioned at the interior of the distal end of the stem, an absorbent material, such as a cotton pad, can be fixed to the exterior of the distal end of the stem. The removable handle member can now be inserted through the proximal end of the stem with its limit reaching the obstructive element at the distal end of the stem. Similarly, more than one disposable swab system can be manufactured at a given time. The solid cylindrical material can be positioned at both ends of the stem, and the desired portion will be introduced at the hollow proximal end and the hollow distal end. When the desired portions are then separated from the rest of the material, an obstructive element is then formed at both ends of the stem, creating a first obstructive end and a second obstructive end. Next, an absorbent material will be affixed to the first obstructive end and the second obstructive end. The stem with the absorbent materials on each end is then split in the middle, by a rotating blade, or another mechanism, resulting in two stems. Each stem will have its obstructive element or plug in the interior of the distal end, with the absorbent material at the exterior of the distal end, and a hollow proximal end. The removable handle member can now be interconnected with the proximal end of the stem and utilized by a user. After utilization, the stem can be disposed and the removable handle member can be interconnected with a new stem.

These and other objects, features, and advantages of the present invention will become clearer when the drawings as well as the detailed description are taken into consideration.

### BRIEF DESCRIPTION OF THE DRAWINGS

For a fuller understanding of the nature of the present invention, reference should be had to the following detailed description taken in connection with the accompanying drawings in which:

FIG. 1A-1D depicts a frontal view of a disposable swab system in accordance with one embodiment of the present invention.

FIG. 2 depicts a frontal view of a stem in an upright position in accordance with the embodiment depicted in FIGS. 1A-1D.

FIG. 3 depicts a front view of a stem with a plug disposed within the interior of its distal end for determining the limits of a removable handle member in accordance with one embodiment of the present invention.

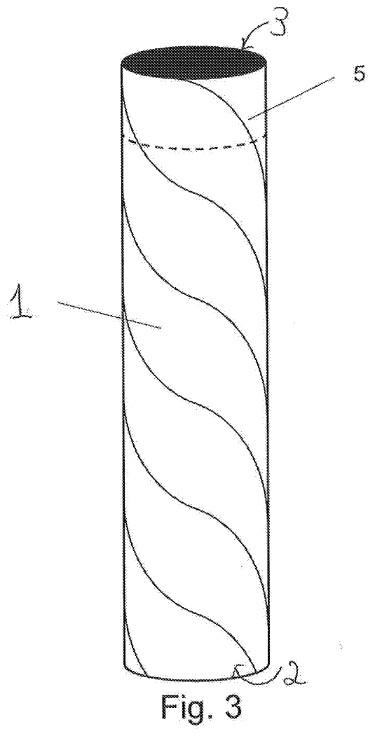

FIG. 4 depicts a removable handle member in accordance with one embodiment of the present invention.

FIG. 5A-5C depicts the removable handle member of FIG. 4 being interconnected with a stem in accordance with one embodiment of the present invention.

FIG. 6A-6C is a frontal view of another embodiment of the present invention with the removable handle member and the stem having an inverse relationship to that represented in FIG. 5A-5C and FIG. 1A-1D.

FIG. 7 is a perspective view of a packaging system, in partial cutaway, for storing and using the disposable swab system in accordance with one embodiment of the present invention.

FIG. 8A-8B depicts a frontal view of one embodiment of the packaging system for storing and using the disposable swab system of the present invention.

FIG. 9A-9D depicts a method for manufacturing a stem of the disposable swab system in accordance with one embodiment of the present invention.

FIG. 10A-10D depicts a method for manufacturing more than one stem at a time of the disposable swab system in accordance with one embodiment of the present invention.

Like reference numerals refer to like parts throughout the several views of the drawings.

### DETAILED DESCRIPTION OF THE PREFERRED EMBODIMENT

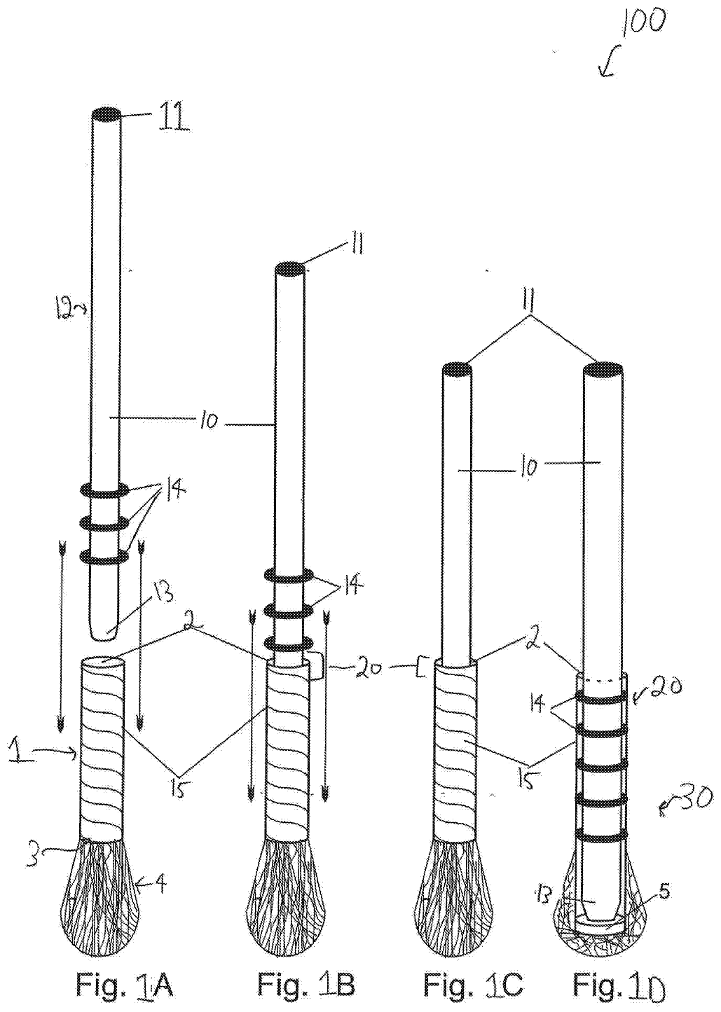

FIG. 1A-1D illustrates one preferred embodiment of a disposable swab system 100. Accordingly, the disposable swab system 100 comprises a stem 1 having a proximal end 2 and a distal end 3. An absorbent material 4, including but not limited to a cotton material, is disposed on the distal end 3. The disposable swab system 100 further comprises a removable handle member 10. As illustrated in FIG. 1B-1D, the removable handle member 10 is dimensioned and configured to be in an interconnected relationship 20 with the proximal end 2 of the stem 1. The interconnected relationship 20 may comprise a mating of the removable handle member 10 and the proximal end 2.

As illustrated in FIGS. 1A-1D, the proximal end 2 may be open and disposed in a communicating relationship with a hollow interior of stem 1. As depicted in FIG. 1B, the removable handle member 10 may be partially or fully inserted through the proximal end 2. The removable handle member 10 comprises a diameter less than a diameter of the stem 1. The removable handle member 10 may also comprise at least one grip ring 14 in retaining engagement with stem 1. FIGS. 1C-1D illustrate the interconnected relationship 20 between the removable handle member 10 and the stem 1. The interconnected relationship 20 allows the disposable swab system 100 to be in an operative position 30. In this embodiment, the operative position 30 necessarily comprises having the removable handle member 10 at least partially disposed within a hollow interior of the stem 1. When in an operative position 30, a user may utilize the disposable swab system 100 for a variety of purposes, including cleaning one's ears and general medical or cosmetic care.

As shown in FIG. 1A-1D, the removable handle member 10 may comprise a circular end 11, a cylindrical body 12, and a conic-shaped end 13. However, the removable handle member 10 may comprise a variety of shapes capable of forming the interconnected relationship 20. In this preferred embodiment, the conic-shaped end 13 of the removable handle member 10 can be inserted into the proximal end 2 of the stem 1. A predetermined portion of the cylindrical body 12 is disposed within stem 1. The predetermined portion of the removable handle member 10 inserted within stem 1 will provide sufficient rigidity and support for the disposable swab 100. The user will then be able to grip the portion of the removable handle member 10 disposed outside of stem 1. A user can now utilize the disposable swab system 100 in the operative position 30.

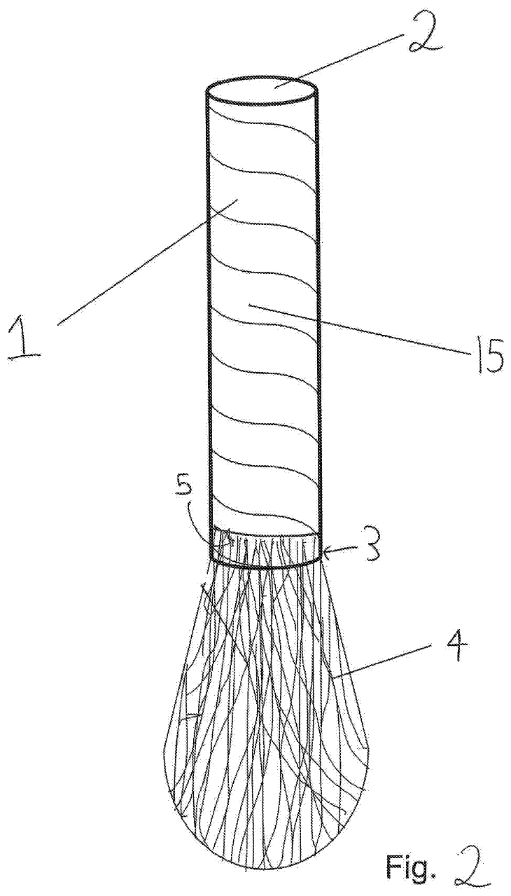

Drawing attention to FIGS. 1A-1D and FIG. 2, stem 1 may be kept in an upright position 15, an easy and accessible position relative to interconnection with the removable handle member 10. The upright position 15 will have the proximal end 2 of the stem 1 facing upwards, with the distal end 3 and the affixed absorbent material 4 facing down. This configuration allows a user to conveniently utilize the removable handle member 10 for proper insertion with stem 1, forming the interconnected relationship 20.

As further illustrated in FIG. 1D, the disposable swab system 100 may also comprise an obstructive element or plug 5 at the interior of the distal end 3 of the stem 1. The plug 5 can serve as a protective structure, functioning as a physical limitation for the reach of the removable handle member 10 when disposed within stem 1. Accordingly, the removable handle member 10 will not surpass the limits of the plug 5 and the absorbent material 4 located at the distal end 3. As such, the disposable swab system 100 has safety features in place to protect a user from any potential misuse when utilizing the disposable swab system 100. For example, when cleaning one's ear(s), navel, or any other areas. The plug 5 may be made of environmentally friendly materials, including cellulose, paper, or any other suitable material. The plug 5 may have a diameter less than an internal diameter of the stem 1.

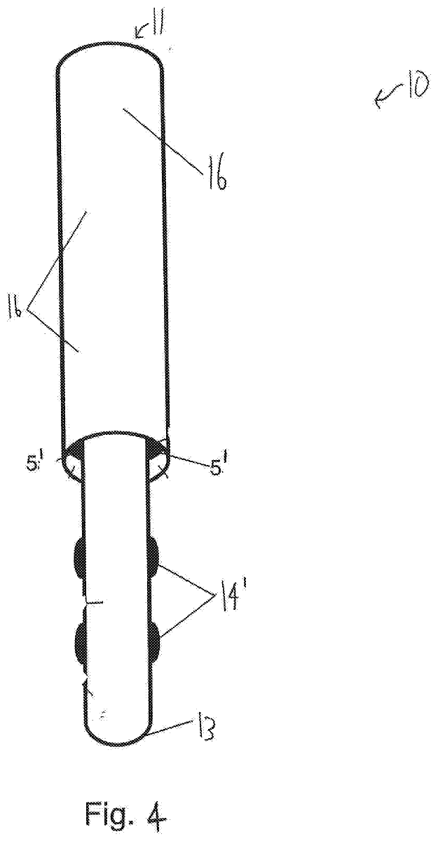

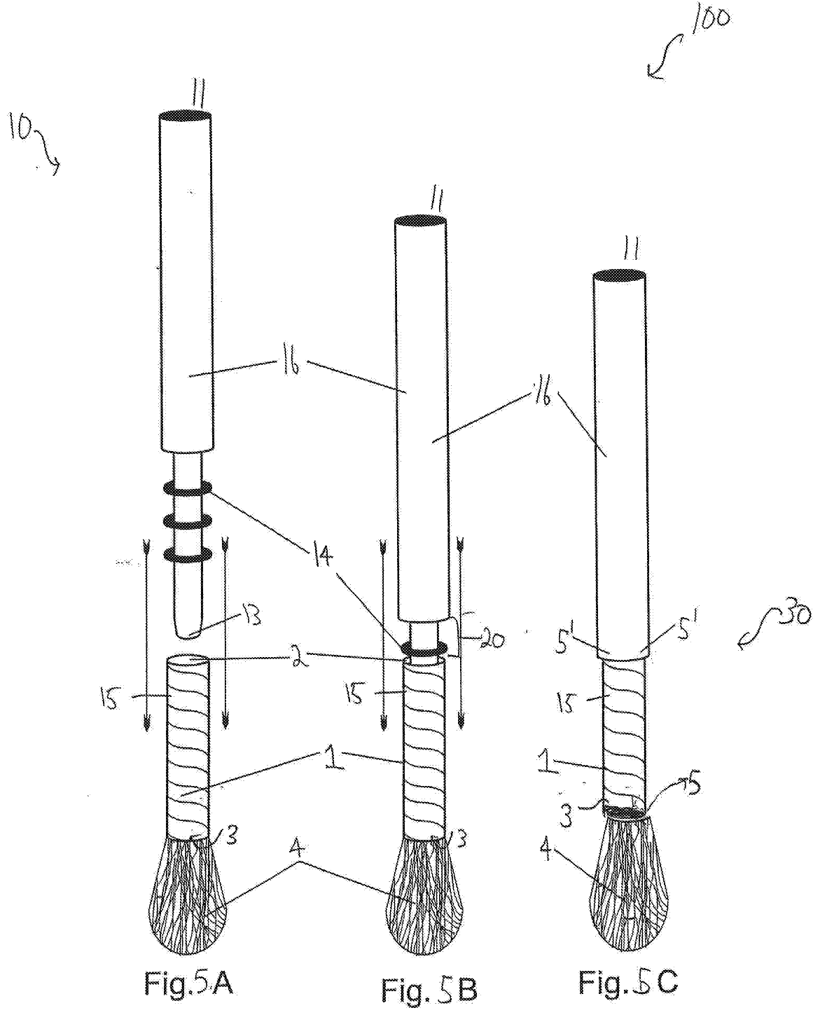

Turning to FIG. 4 and FIGS. 5A-5C, another embodiment of the removable handle member 10 is depicted. The removable handle member 10 may comprise a larger, thicker cover structure 16 at its upper end towards the circular end 11. The bottom end of the cover structure 16, toward the conic-shaped end 13, may also act as another obstructive element which may be in the form of a shoulder structure 5′. The shoulder structure 5′ will act as a second limitation to the reach of the removable handle member 10 to prevent any potential misuse of the disposable swab system 100 when in an operative position 30. This is illustrated in FIG. 5C when the removable handle member is disposed within stem 1. The conic-shaped end 13 will be introduced into the proximal end 2, disposed in an upright position 15, and the shoulder structures 5′ at the bottom end of the cover structure 16 will act as a limitation to the reach of the removable handle member 10 within the stem 1. Similar to plug 5, the shoulder structures 5′ will protect users when utilizing the disposable swab system 100.

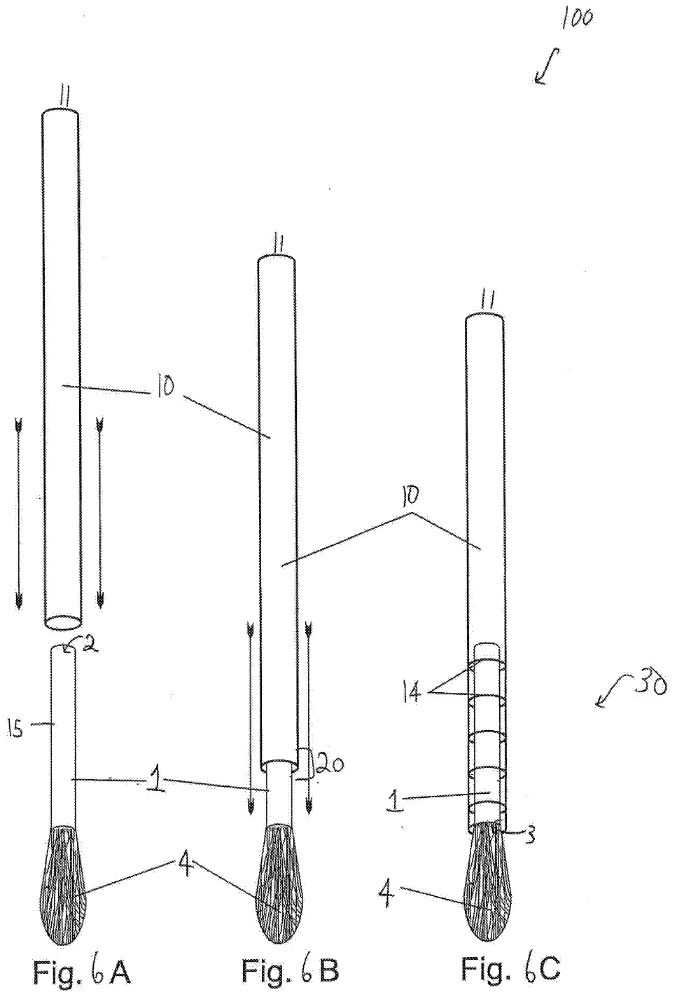

Drawing your attention to FIGS. 6A-6C, a further embodiment of the disposable swab system 100 is shown. Accordingly, FIGS. 6A-6C depict an inverse relationship between stem 1, with the affixed absorbent material 4, and the removable handle member 10. In this embodiment, stem 1 will have a diameter less than a diameter of the removable handle member 10. Stem 1 may be at least partially solid, while the removable handle member 10 may be at least partially hollow.

As illustrated in FIGS. 6B-6C, the removable handle member 10 can be disposed around the stem 1. Similarly, the proximal end 2 of the stem 1 will be inserted within the removable handle member 10, providing the necessary means of accomplishing the interconnected relationship 20. In this embodiment, the operative position 30 comprises stem 1 being disposed within a hollow interior of the removable handle member 10. Additionally, as illustrated in FIG. 6C, the removable handle member 10 may comprise at least one grip ring 14 located on its interior structured in retaining engagement with stem 1. In this embodiment, the stem 1, being at least partially solid and disposed within the removable handle member 10, will provide the rigidity and density to be conveniently utilized when the removable handle member 10 is in an operative position 30.

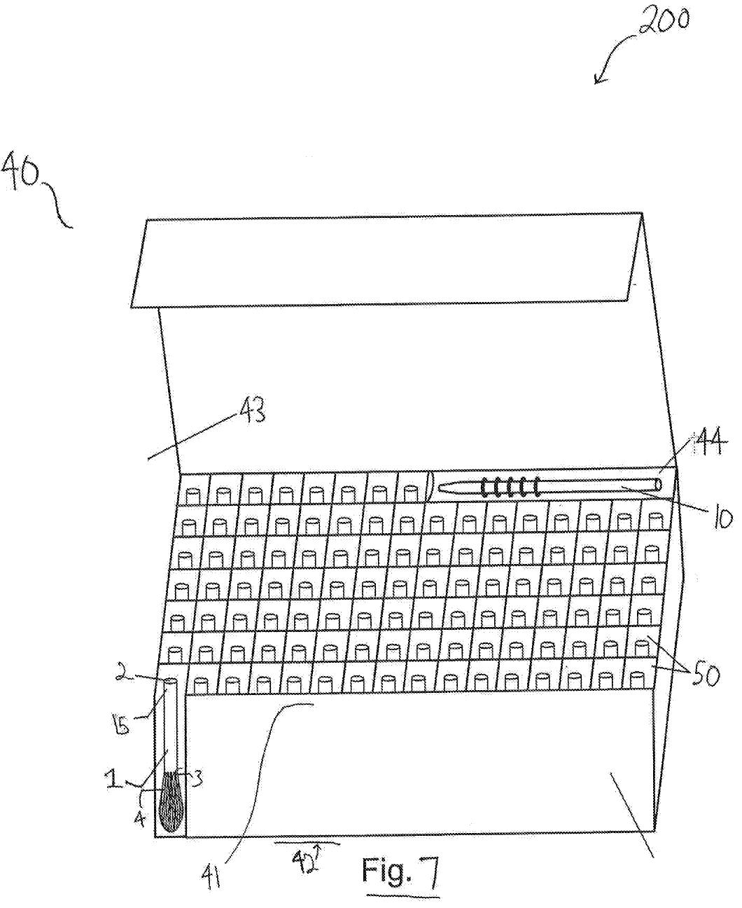

The present invention can also be incorporated into a packaging system 200 for storing and using the disposable swab system 100 by a user. As illustrated in FIG. 7, in one preferred embodiment of the packaging system 200 for storing and using the disposable swab system 100, the system 200 comprises a container 40. The container comprises an open top 41 and a closed bottom 42, and a plurality of individual housing compartments 50 for storing a plurality of stems 1. The system 200 may also comprise a cover 43 movably disposed in covering relation to the open top 41. The packaging system 200 will also comprise a plurality of stems 1 having a proximal end 2 and a distal end 3, with an absorbent material fixed at each distal end 3 of the plurality of stems 1. The plurality of stems 1 will be housed within the individual compartments 50. The individual compartments 50 may have a diameter greater than a diameter of the stems 1 for proper storage. The packaging system 200 will also comprise the removable handle member 10, which may be housed in a separate compartment 44 within the container 40.

Importantly, the plurality of individual compartments 50 are configured to house a different one of the plurality of stems 1 in an upright position 15. The upright position 15 comprises the proximal end 2 being disposed adjacent to the open top 41 in an exposed, accessible position relative to interconnection with the removable handle member 10. The distal end 3 and absorbent material 4 will be disposed adjacent to the closed bottom 42. The upright position 15 of the stem 1 may allow a user to easily introduce the removable handle member 10 into the proximal, hollow end 2 of stem 1, without having to first remove the stem 1 from the container 40. Accordingly, the system 200 provides an easy configuration for inserting the removable handle member 10 into a stem 1, completing the interconnected relationship 20 and allowing a user to utilize the disposable swab system 100. The user can then dispose of the stem 1, and re-insert the removable handle member 10 into the next stem 1 housed in the plurality of individual compartments 50.

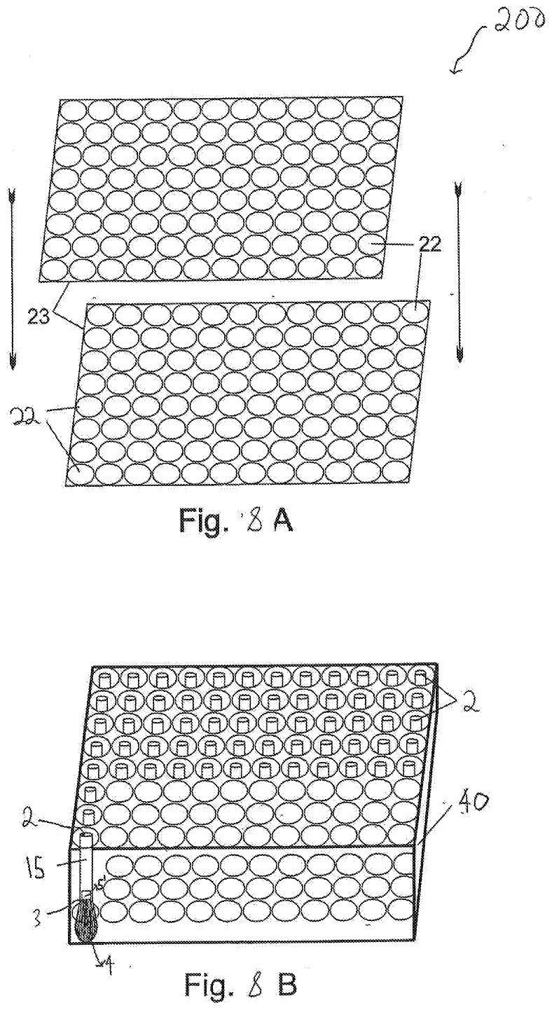

FIGS. 8A and 8B show a constructive model of the grooves 22 that comprise the individual compartments 50, for keeping the stems 1 in an upright position 15. The grooves 22 may be in any particular shape, including a circular shape, which may form a two-layer structure 23 for housing the stems 1. The grooves 22 will comprise a diameter and length suitable to accommodate the stems' 1 diameter. The two-layer structure 23 of grooves 22 may be made up of a carton material, or other material. This will provide for easy and comfortable utilization of both the packaging and use system 200, and the utilization of the disposable swab system 100. In line with the environmental objectives of the present invention, the packaging system 200 may be composed of environmentally friendly materials, such as cellulose material, carton material, recyclable material, or a variety of other materials.

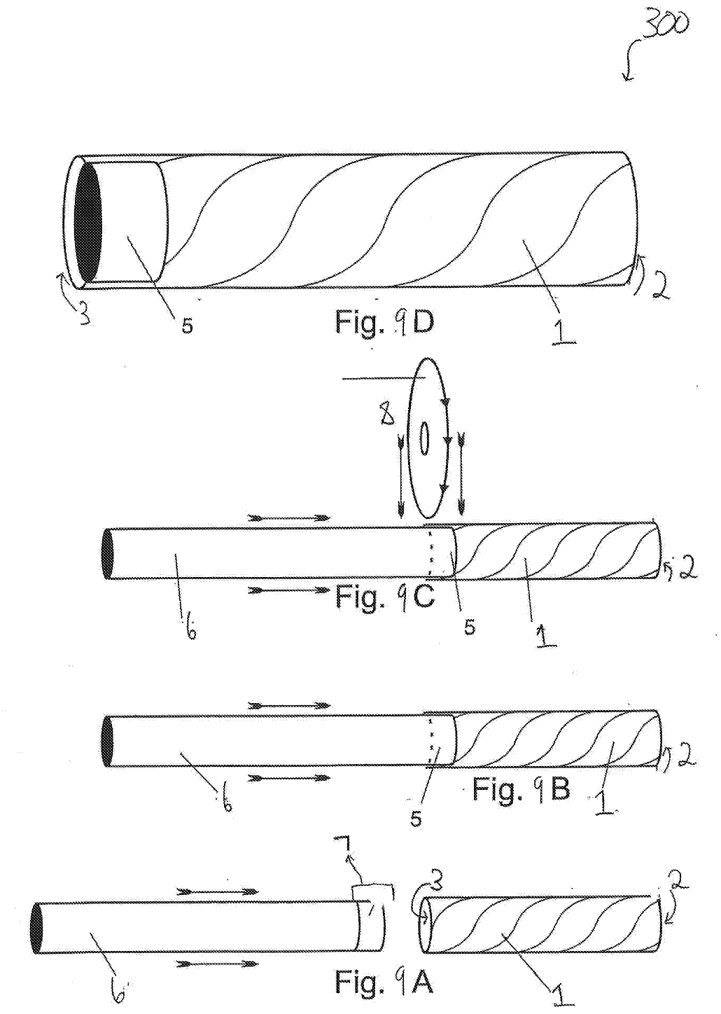

The present invention also incorporates a method 300 for manufacturing the stem 1 of the disposable swab system 100. Accordingly, as illustrated in FIG. 9A-9D, an important step in this method 300 is to form an obstructive element, or plug 5 within the distal end 3 of the stem 1. The plug 5 functions as a protective limitation for the removable handle member's 10 reach when introduced into the proximal end 2 of stem 1, when in an interconnected relationship 20. The plug 5 may have an external diameter less than the internal diameter of the stem 1. The plug 5 may be composed of a variety of materials, but in one embodiment, environmentally friendly materials such as paper or cellulose material are used. Accordingly, as depicted in FIG. 9A, a long, continuous, solid cylindrical material 6 will be set in a direct, confronting relation relative to the stem 1, so that a desired portion 7 of the solid cylindrical material 6 will be introduced into the hollow, distal end 3 of the stem 1. The solid cylindrical material 6 may comprise cellulose materials, including but not limited to paper. As depicted in FIG. 9B, the predetermined or desired portion 7 of the solid cylindrical material 6 will now obstruct the distal end 3 of the stem 1, creating the plug 5. The plug 5 can be separated from the rest of the solid cylindrical material 6, situating plug 5 at the distal end 3. The separation can be accomplished by a rotative blade 8 or by other means. Plug 5 may take the shape of a disc, or any other shape that can be accommodated within the stem 1. Now that the obstructive element and/or plug 5 is firmly positioned at the interior of the distal end 3, an absorbent material 4 can be fixed at the exterior of distal end 3 of stem 1.

The removable handle member 10 can now be inserted through the proximal end 2 of the stem 1, with its limits reaching the plug 5 at the distal end 3 of the stem 1, protecting the user from any potential misuse.

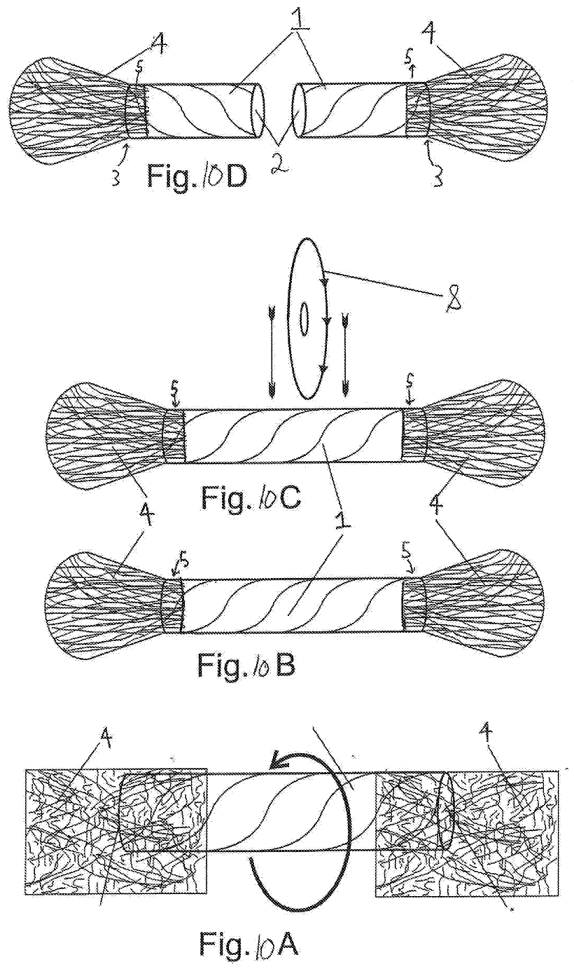

Additionally, as illustrated in FIGS. 10A-10D, more than one disposable stem 1 can be manufactured at a given time. As was illustrated in FIG. 9A-9D, the solid cylindrical material 6 is positioned at the distal end 3 of the stem 1. Similarly, a plurality of solid cylindrical material 6 can be positioned at both ends of the stem 1. The desired portions 7 can be introduced at both the hollow proximal end 2 and the hollow distal end 3. When the predetermined or desired portions 7 are separated or cut from the rest of the cylindrical material 6, an obstructive element or plug 5 will be fixed or formed at both ends of the stem 1, creating two obstructive ends. Now, as illustrated in FIG. 10B, the absorbent material 4 can be attached to each end of the stem 1 with both ends having a plug 5 disposed therein. The stem 1 can now be split or cut, resulting in two stems 1, which may be accomplished by a rotative blade 8 or other means. The cutting of stem 1 will occur in between the obstructive ends, preferably equidistant to both obstructive ends. Accordingly, each stem 1 will comprise a plug 5 disposed in the interior of distal end 3, an absorbent material 4 at the exterior of distal end 3, and a hollow proximal end 2. The removable handle member 10 can now be introduced and mated with stem 1, forming the interconnected relationship 20.

Since many modifications, variations and changes in detail can be made to the described embodiments of the invention, it is intended that all matters in the foregoing description and shown in the accompanying drawings be interpreted as illustrative and not in a limiting sense. Thus, the scope of the invention should be determined by the appended claims and their legal equivalents.

What is claimed is:

1. A disposable swab system, comprising:
a stem having a proximal end and a distal end,
a removable handle member dimensioned and configured to be disposed in an interconnected relationship to said proximal end of said stem when in an operative position, said interconnected relationship comprising a mating of said removable handle member and said proximal end of said stem,
said stem further comprising an absorbent material disposed on an exterior of said distal end.

2. The disposable swab system as recited in claim 1 wherein said removable handle member further comprising at least one grip ring disposed and structured in retaining engagement with said stem.

3. The disposable swab system as recited in claim 1 wherein said proximal end is open and disposed in communicating relation with a hollow interior of said stem.

4. The disposable swab system as recited in claim 3 wherein said removable handle member has a diameter less than a diameter of said stem, said operative position comprising said removable handle member at least partially disposed within said hollow interior of said stem, through said proximal end.

5. The disposable swab system as recited in claim 3 further comprising an obstructive element.

6. The disposable swab system as recited in claim 5 wherein said obstructive element comprises a plug disposed at an interior of said distal end of said stem.

7. The disposable swab system as recited in claim 5 wherein said obstructive element comprises a shoulder structure at an exterior of said removable handle member.

8. The disposable swab system as recited in claim 1 wherein said stem has a diameter less than a diameter of said removable handle member, said operative position comprising said stem disposed within a hollow interior of said removable handle member.

9. The disposable swab system as recited in claim 8 wherein said stem is at least partially solid.

10. The disposable swab system as recited in claim 1 wherein said stem comprises a cellulose material.

11. The disposable swab system as recited in claim 1 wherein said absorbent material comprises a cotton material.

12. A packaging system for storing and using a disposable swab system by a user, said packaging system comprising:
a container comprising an open top, a closed bottom, and a plurality of individual compartments,
a plurality of stems comprising a proximal end and a distal end,
said plurality of stems comprising an absorbent material fixed at said distal end,
a removable handle member,
said plurality of individual compartments configured to house a different one of said plurality of stems, said plurality of stems disposed in said plurality of individual compartments in an upright position, and
said upright position comprising said proximal end disposed adjacent said open top in an exposed, accessible position relative to interconnection with said removable handle member.

13. The packaging system as recited in claim 12 wherein said container further comprises a cover movably disposed in covering relation to said open top.

14. The packaging system as recited in claim 12 wherein said removable handle member dimensioned and configured to be disposed in an interconnected relationship to said proximal end of said different one of said plurality of stems when in an operative position, said interconnected relationship comprising a mating of said removable handle member and said proximal end.

15. The packaging system as recited in claim 14 wherein said removable handle member has a diameter less than a diameter of said different one of said plurality of stems, said operative position comprising said removable handle member at least partially disposed within a hollow interior of said different one of said plurality of stems, through said proximal end.

16. The packaging system as recited in claim 12 wherein said removable handle member further comprising at least one grip ring disposed and structured in retaining engagement with said different one of said plurality of stems.

17. The packaging system as recited in claim 12 wherein each of said plurality of stems comprises an obstructive element, said obstructive element comprises a plug disposed at an interior of said distal end.

18. The packaging system as recited in claim 12 wherein said plurality of stems comprise a cellulose material.

19. The packaging system as recited in claim 12 wherein said absorbent material comprises a cotton material.

20. A method for manufacturing and using at least one stem of the disposable swab system, said method comprising:
positioning at least one solid cylindrical material in a direct, confronting relation with a stem having a proximal end and a distal end,
introducing a predetermined portion of the at least one solid cylindrical material in the proximal end and the distal end of the stem,
cutting the predetermined portion of the at least one solid cylindrical material in the proximal end and the distal end of the stem, forming a first obstructive end and a second obstructive end of the stem,
applying an absorbent material to the first obstructive end and the second obstructive end of said stem,
cutting the stem in between the first obstructive end and the second obstructive end,
producing at least one stem having a hollow, proximal end and an obstructive distal end, the obstructive distal end having the absorbent material attached thereon.

21. The method as recited in claim 20 further comprising concentrically mating a removable handle member through the hollow, proximal end of the at least one stem.

22. The method as recited in claim 21 further comprising disposing the at least one stem, and repeating the concentrically mating step of the removable handle member.
